(19) United States
(12) Patent Application Publication
(10) Pub. No.: US 2024/0245424 A1
(43) Pub. Date: Jul. 25, 2024

(54) JOINT ASSEMBLY FOR VASCULATURE OBSTRUCTION CAPTURE DEVICE

(71) Applicant: Neuravi Limited, Galway (IE)

(72) Inventors: Brendan CASEY, Galway (IE); David VALE, Barna (IE); Maeve HOLIAN, Galway (IE); Brian FAHY, Galway (IE)

(73) Assignee: Neuravi Limited, Galway (IE)

(21) Appl. No.: 18/624,408

(22) Filed: Apr. 2, 2024

Related U.S. Application Data
(62) Division of application No. 17/879,914, filed on Aug. 3, 2022, now Pat. No. 11,963,693.
Application No. 17/879,914 is a division of application No. 16/150,024, filed on Oct. 2, 2018, now Pat. No. 11,406,416.

(51) Int. Cl. A61B 17/3205; A61B 17/00; A61B 17/221; A61M 25/00

(52) U.S. Cl. CPC A61B 17/3205; A61B 17/221; A61B 2017/00477; A61M 25/0043

(57) ABSTRACT

A joint assembly for an endovascular device, comprising a shaft comprising a main body and an enlarged end, a proximal strut comprising a slot, wherein the slot engages the enlarged end of the shaft and a locking collar comprising a proximal face and a distal face, and at least partially covering the enlarged end of the shaft and the slot of the proximal strut, such that the locking collar constrains the proximal strut such that the proximal strut cannot disengage from the enlarged end of the shaft when the joint assembly and the clot retrieval device is under load.

### CROSS REFERENCE TO RELATED APPLICATION

The present application is a divisional application of U.S. patent application Ser. No. 17/879,914, filed Aug. 3, 2022, which is a divisional of Ser. No. 16/150,024 filed Oct. 2, 2018, now issued as U.S. Pat. No. 11,406,416 on Aug. 9, 2022. The entire contents of each of which are hereby incorporated by reference.

### FIELD OF INVENTION

The present invention generally relates to endovascular devices and methods, and, more particularly, to endovascular joint assemblies which can be used as components of endovascular devices used to remove blood vessel obstructions.

### BACKGROUND

Recent clinical studies have shown that mechanical thrombectomy is an increasingly effective method of acute obstruction removal from blood vessels. Acute obstructions can include clots, misplaced devices, migrated devices, large emboli and the like. An ischemic stroke can result if an obstruction lodges in the cerebral vasculature. A pulmonary embolism can result if the obstruction, such as a clot, originates in the venous system or in the right side of the heart and lodges in a pulmonary artery or branch thereof. Mechanical thrombectomy typically involves advancing a thrombectomy device or stentriever to the occlusive clot, engaging with the clot and retracting the clot into the safety of a proximally placed guide or sheath.

However, despite the benefits provided by mechanical thrombectomy devices, there are limitations. For example, there are a number of procedural challenges that can place undue tension or compression on the device components. In cases where access involves navigating the aortic arch (such as coronary or cerebral blockages) the configuration of the arch in some patients makes it difficult to position a stentriever. These difficult arch configurations are classified as either type 2 or type 3 aortic arches with type 3 arches presenting the most difficulty. The tortuosity challenge is even more severe in the arteries approaching the brain. For example, it is not unusual at the distal end of the internal carotid artery that the device will have to navigate a vessel segment with a 180° bend, a 90° bend and a 360° bend in quick succession over a few centimeters of the vessel. Delivering the device through the tortuous anatomy to the target location can apply compressive loading on the device components and joint between the distal section and the shaft. Moreover, dislodgement force of the obstruction in the vessel and retrieval through the tortuosity of the vasculature can place high tensile loading on the joint. Retrieval of the obstruction into the access catheter can also place high forces on the device components and proximal joint to the shaft.

These endovascular devices can be integrally formed with joint assemblies, often connecting a clot engaging portion to an elongated shaft. These assemblies can rely on adhesive or weld bonds, or soldering the joint. Adhesive can be applied to ensure the components maintain the correct position and orientation but increased joint strength and integrity can be desirable in some instances. As shown in FIG. 1, a previously disclosed joint assembly can include a shaft 10 including a main body 12 and an enlarged step 14, a proximal strut 18 engaging with shaft 10, and a locking collar 16 engagingly receiving at least a portion of main body 12 and at least a portion of proximal strut 18 to lock the assembly into place. However, as shown in FIG. 2, undue tension can elicit so much tensile stress on the shaft, that the proximal strut disengages from the enlarged step of the shaft, and the enlarged end deforms. This can result in disassembly of the joint device of the stentriever or thrombectomy device during dislodgement of the obstruction or as it is withdrawn proximally around a bend in a tortuous vessel, or the potential escape of the captured clot.

There therefore exists a need for an endovascular device with sufficient integrity for effectively capturing an obstruction for safe retrieval from a patient.

### SUMMARY

Disclosed herein are various exemplary devices of the present invention that can address the above needs, the devices can be joint assemblies that generally can include a shaft, a proximal strut, and a locking collar. The joint assemblies can be integrally joined to an endovascular device, between a clot engaging portion and an elongated shaft. In this manner, the joint assemblies permit for an obstruction to be captured by the clot engaging portion of an endovascular device with increased load support provided by the joint assemblies.

In one example, the joint assembly for an endovascular device can include a shaft having a main body and an enlarged end, a proximal strut comprising a slot, wherein the slot engages the enlarged end of the shaft, and a locking collar including a proximal face and a distal face, and at least partially covering the enlarged end of the shaft and the slot of the proximal strut. In some embodiments, at least a portion of the enlarged end is received in the proximal strut slot. In some embodiments, the enlarged end of the shaft defines a shaft step with the main body of the shaft.

In another example, the joint assembly for an endovascular device can include a shaft with a main body and an enlarged end, a proximal strut with a slot, wherein the main body of the shaft is received in the slot, and a locking collar having a proximal face, a distal face, and at least partially covering the enlarged end of the shaft and the slot of the proximal strut. In some embodiments, the proximal strut slot can include a first height and a second height. In some embodiments, the main body of the shaft is inserted into the proximal strut slot at the first height, the enlarged end engages the proximal strut slot at the second height, and the proximal strut slot slopes downwards from the second height and away from the enlarged end.

In another example, the joint assembly for an endovascular device can include a shaft with a main body and an enlarged end, a proximal strut having a cross-shaped member, and a locking collar with a proximal face, a distal face, and at least partially covering the enlarged end of the shaft and the slot of the proximal strut. In some embodiments, the cross-shaped member engages the distal face of the locking collar.

In another example, a method of assembling the joint assembly can include the steps of providing a shaft with a main body and an enlarged end, a proximal strut including a slot, wherein the slot engages the enlarged end of the shaft, and a locking collar has a proximal face and a distal face and engaging the enlarged end of the shaft and the slot of the proximal strut, sliding the locking collar onto the main body of the shaft, positioning at least a portion of the proximal strut proximate the enlarged end of the shaft, and repositioning the collar such that it at least partially covers the enlarged end of the shaft and the slot of the proximal strut. In some embodiments, when the locking collar is repositioned, the collar constrains the proximal strut such that the proximal strut slot cannot disengage from the enlarged end of the shaft when the joint assembly is loaded into an endovascular device and the endovascular device is under load.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 illustrates configurations of an exemplary prior art joint assembly;

FIG. 2 illustrates configurations of an exemplary prior art joint assembly under tensile load;

FIG. 3 illustrates a perspective view of an exemplary joint assembly of the present invention;

FIGS. 4A to 4B illustrate a side and end view of an exemplary locking collar of a joint assembly of the present invention;

FIGS. 5A to 5C illustrate a method of assembly of a joint assembly of the present invention;

FIGS. 6A to 6D illustrate configurations of an exemplary joint assembly of the present invention;

FIGS. 7A to 7D illustrate configurations of an exemplary joint assembly of the present invention;

FIGS. 8A to 8C illustrate configurations of an exemplary joint assembly of the present invention;

FIGS. 9A to 9B illustrate a side view of an exemplary joint assembly of the present invention;

FIGS. 10A to 10C illustrate configurations of an exemplary joint assembly of the present invention;

FIG. 11 illustrates a perspective view of an exemplary joint assembly of the present invention; and

FIGS. 12A to 12B illustrate perspective views of an exemplary joint assembly integrally formed into an endovascular device.

### DETAILED DESCRIPTION

Specific embodiments of the present invention are now described in detail with reference to the figures, wherein identical reference numbers indicate identical or functionality similar elements. The terms “distal” or “proximal” are used in the following description with respect to a position or direction relative to the treating physician. “Distal” or “distally” are a position distant from or in a direction away from the physician. “Proximal” or “proximally” or “proximate” are a position near or in a direction toward the physician.

Accessing cerebral, coronary and pulmonary vessels involves the use of a number of commercially available products and conventional procedural steps. Access products such as stentrievers and thrombectomy devices are described elsewhere and are regularly used in endovascular procedures. It is assumed in the descriptions below that these products and methods are employed in conjunction with the device and methods of this invention and do not need to be described in detail.

The following detailed description is merely exemplary in nature and is not intended to limit the invention or the application and uses of the invention. Although the description of the invention is in many cases in the context of treatment of blood vessel occlusions, the invention may also be used in other body passageways as described herein.

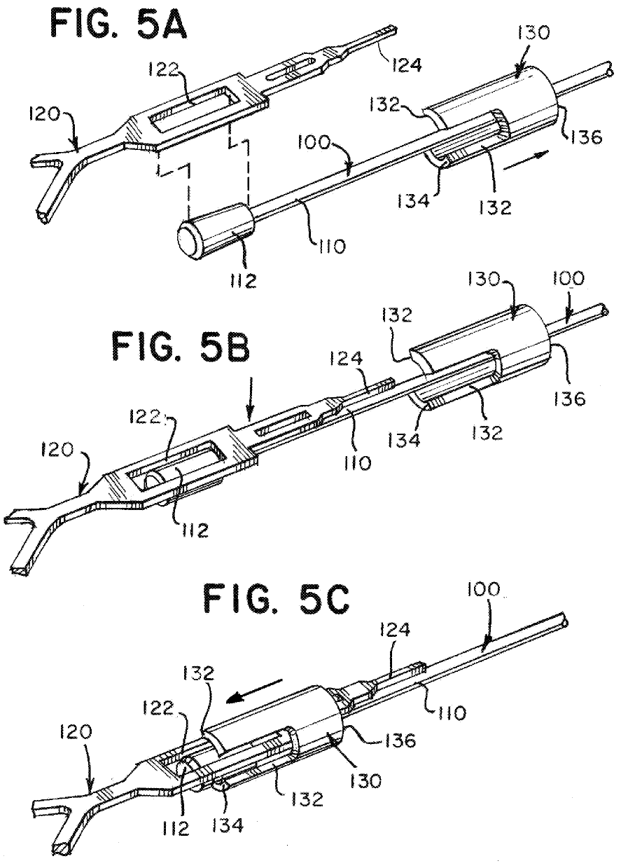

An example of a joint assembly, as illustrated in FIG. 3 can have a shaft 100, a proximal strut 120, and a locking collar 130. The proximal strut 120 is distal of the shaft but at the proximal end of the stentriever (see FIG. 12A). In some embodiments, the shaft 100 can include a main body 110 and an enlarged end 112. In some embodiments, the proximal strut 120 can include a slot 122. In some embodiments, the slot 122 can engage the enlarged end 112 of the shaft 100. In some embodiments, the locking collar 130 can at least partially cover the enlarged end 112 of the shaft 100 and the slot 122 of the proximal strut 120. In some embodiments, at least a portion of the enlarged end 112 is received in the proximal strut slot 122. In some embodiments, the enlarged end 112 of the shaft 100 defines a shaft step with the main body 110 of the shaft 100. In some embodiments, proximal strut further includes a tail 124 as shown in FIG. 5A. In some embodiments, the slot 122 of the proximal strut 120 or a secondary slot can be adjacent to tail 124 and facilitate the wicking of adhesive into the proximal strut 120 to maintain the proper orientation of the joint assembly components.

Suitable materials for forming the shaft, proximal strut, and collar ideally have a high tensile strength such that sufficient integrity for manufacturability and use can be produced, such as for example polymers materials like UHMWPE, Aramid, LCP, PET or PEN, or metals such as Tungsten, MP35N, stainless steel or Nitinol. Proximal strut slot 122 can be any suitable shape for engaging enlarged end 112.

In some embodiments, the joint assembly can be any suitable size and shape to be compatible with microcatheters used for neurovascular device delivery. Suitable shapes for the proximal strut slot 122 can include generally square, generally rectangular, generally circular, and the like. Locking collar 130 can be any suitable shape for covering or enclosing at least a portion of the proximal strut slot 122 and the enlarged end 112 of shaft 100. Suitable shapes for locking collar 130 can include generally cylindrical, generally elliptical cylindrical, and the like. Main body 110 and enlarged end 112 of shaft 100 can be any suitable size and shape for engaging proximal strut 120 and being received, at least partially, in locking collar 130. Suitable shapes for main body 110 can include generally cylindrical, generally elliptical cylindrical, and the like. Suitable shapes for enlarged end 112 can include generally cylindrical, generally elliptical cylindrical, and the like. In some embodiments, the joint assembly can be sized to be compatible with microcatheters with an inner diameter of 0.027 inches or less (e.g. 0.026 inches, 0.024 inches, 0.022 inches, 0.019 inches, 0.017 inches, 0.015 inches, 0.013 inches, 0.011 inches, 0.009 inches, 0.007 inches, 0.005 inches, 0.003 inches, 0.001 inches), and preferably with a microcatheter having an inner diameter of 0.021 inches or less (e.g. 0.019 inches, 0.017 inches, 0.015 inches, 0.013 inches, 0.011 inches, 0.009 inches, 0.007 inches, 0.005 inches, 0.003 inches, 0.001 inches).

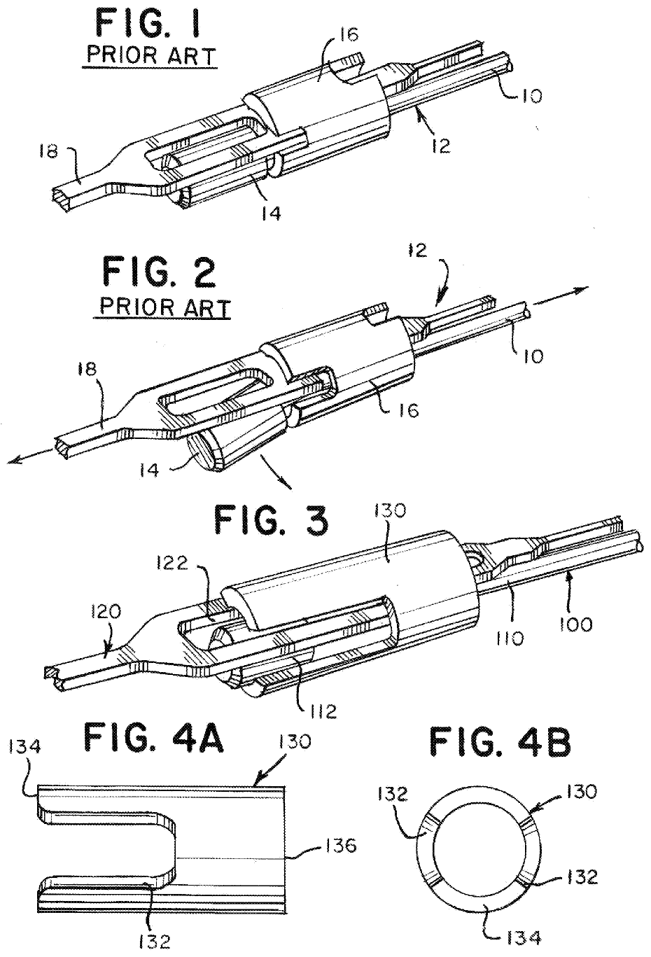

As shown in FIGS. 4A and 4B, in some embodiments, the locking collar 130 can include a proximal face 136 and a distal face 134. In some embodiments, the locking collar can include first and second slits 132. As shown in FIGS. 5A-5C, in some embodiments, assembly of the joint assembly may include providing shaft 100, proximal strut 120, and locking collar 130. As shown in FIG. 5A, the assembly can further include sliding locking collar 130 onto the main body 110 of the shaft 100 away from the enlarged end 112. As shown in FIG. 5B, the assembly can further include positioning at least a portion of proximal strut 120 proximate enlarged end 112 of shaft 100. As shown in FIG. 5C, the assembly can further include repositioning locking collar 130 such that it at least partially covers enlarged end 112 of shaft 100 and slot 122 of proximal strut 120. In some embodiments, when the locking collar is repositioned, the locking collar 130 at least partially encloses the enlarged end 112 of the shaft 100 and the slot 122 of the proximal strut 120. As a result of this configuration, when the joint assembly is under tension the locking collar 130 supports the enlarged end 112 minimizing the deformation illustrated in the previously disclosed joint in FIG. 2. This makes the joint assembly more robust and capable of withstanding higher tensile loads than the prior design.

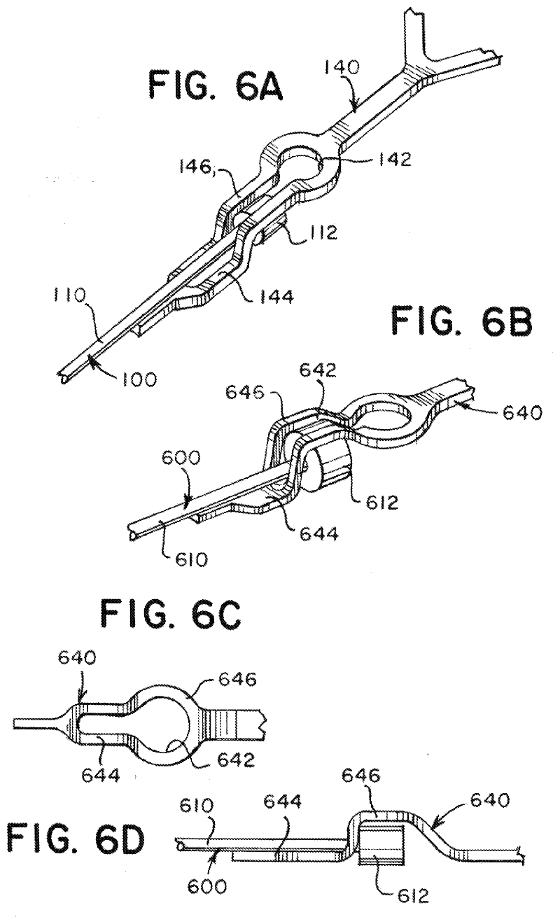

As shown in FIG. 6A, in some embodiments, proximal strut 140 can include a first height 144 and a second height 146. In some embodiments, the main body 110 of the shaft 100 is inserted into the proximal strut slot 142 at the first height 144, and the enlarged member 112 engages the proximal strut slot 142 at the second height 146. As shown in FIGS. 6B-6D, in some embodiments, proximal strut 640 includes a proximal strut slot 642 including a first height 644 a second height 646. In some embodiments, shaft 600 can include main body 610 and enlarged end 612, such that the shaft 600 is inserted into the proximal strut slot 642 at the first height 644, the proximal strut slot 642 engages the enlarged member 612 at the second height 146 and tapers downward and away from the first height 644. The taper assists the proximal strut 640 to engage with the distal end of the shaft enlarged end 612. This increases the tensile and compressive force that may be transferred through this joint assembly from the shaft 600 to the distal end of the joint assembly. The locking collar 130 constrains the proximal strut 122 relative to enlarged end 112 such that proximal strut 122 maintains engagement with enlarged end 112 under tensile load up to a force of from about 2N to 15N (e.g. 3N, 4N, 5N, 6N, 7N, 8N, 9N, 10N, 11N, 12N, 13N, 14N).

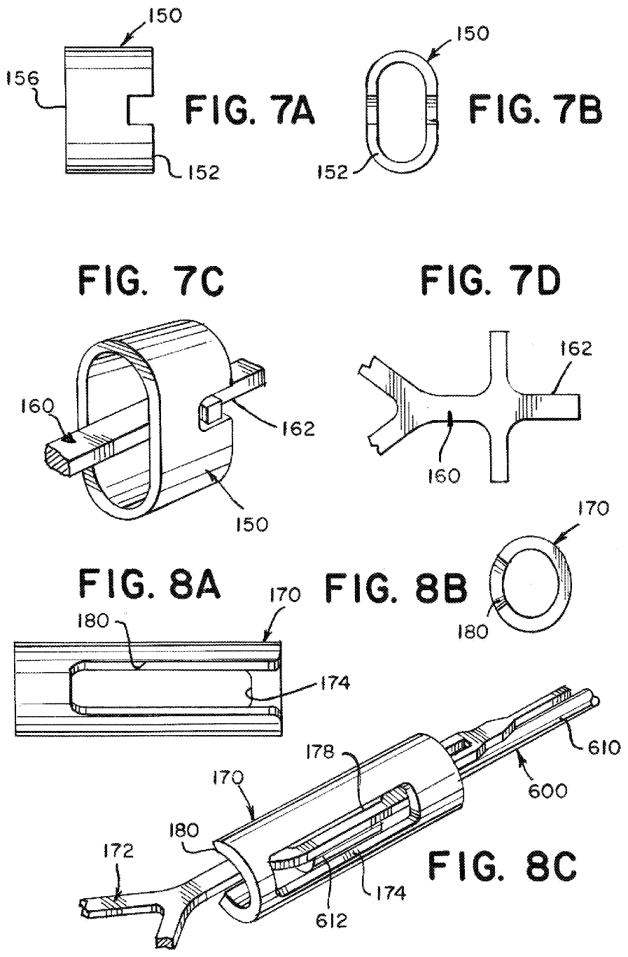

As shown in FIGS. 7A-7D, locking collar 150 can further include a proximal face 152 and a distal face 156, and proximal strut 160 can include a cross-shaped member 162. In some embodiments, the cross-shaped member 162 can engage the proximal face 152 of locking collar 150. In some embodiments, the cross-shaped member 162 can lockingly engage the proximal face 152 of locking collar 150. The cross-shaped member 162, in some examples, can be used to prevent the proximal strut 160 from rotating or provide additional locking support. In certain examples, the locking collar 150 can have an ovoid shape and the cross-shaped member 162 is passed through the locking collar 150 from the distal face 156 to the proximal face 152, as the major axis of the oval is larger than arms of the cross. The cross-shaped member 162 is then rotated 90° to engage the slits on the proximal face 152. In this way, the minor axis of the oval is shorter than the arms of the cross. In some embodiments, this joint assembly could further include a shaft and an enlarged end (not shown in figures).

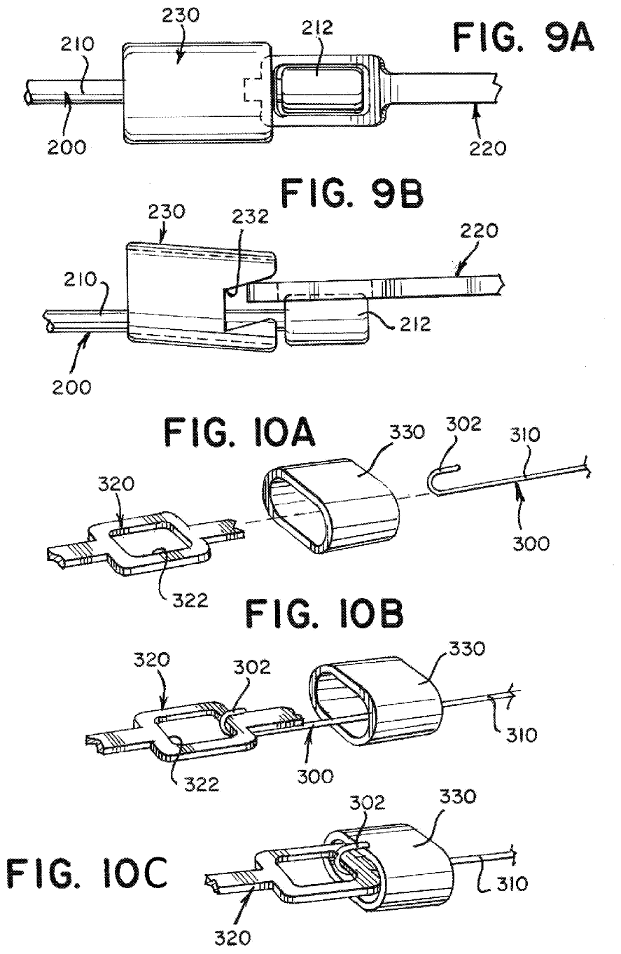

As shown in FIGS. 8A-8C, in some embodiments, locking collar 170 can include a first slit 180 formed in proximal face of locking collar 170 and a polygonal aperture 174 positioned opposite the first slit 180. In some embodiments, at least a portion of the proximal strut 172 may engage the first slit 180. In some embodiments, at least a portion of the proximal strut can engage polygonal aperture 174. In some embodiments, the portion of the proximal strut 172 surrounding the proximal strut slot 178 can engage first slit 180 and polygonal aperture 174. In some embodiments, the portion of the proximal strut 172 surrounding the proximal strut slot 178 can lockingly engage first slit 180 and polygonal aperture 174. In some embodiments, the first slit 180 can be formed from the distal face of the collar 170 into at least a portion of the first side of the collar 170 and a polygonal aperture 174 is formed in the second side of the collar 170. This design can minimize deformation of the locking collar 170 when the exemplary joint assembly is subjected to high tensile forces.

As shown in FIGS. 9A and 9B, in some embodiments, an exemplary joint assembly can include a shaft 200 including a main body 210 and an enlarged end 212, a proximal strut 220, and a locking collar 230 including first and second slits 232. In some embodiments, first and second slits 232 can be tapered. In some embodiments, proximal strut 220 can engage first and second tapered slits 232. In some embodiments, the locking collar 230 can abut the enlarged end 212 of the shaft 200. In some embodiments, the proximal strut 220 can engage the tapered slits 232 of locking collar 230 such that locking collar 230, shaft 200, and proximal strut 220 are lockingly engaged. In some embodiments, the proximal strut 220 can engage the tapered slits 232 of locking collar 230 such that locking collar 230, shaft 200, and proximal strut 220 are lockingly engaged by friction-fit. In some embodiments, the proximal strut 220 can engage the tapered slits 232 of locking collar 230 such that locking collar 230, shaft 200, and proximal strut 220 are lockingly engaged by friction-fit, such that the proximal strut 220 cannot disengage from the enlarged end 212 of the shaft 200 when the joint assembly is integrally formed into an endovascular device and the endovascular device is under load. The taper of the slits can compensate for component dimensional tolerances during assembly and can ensure the proximal strut 220 stays in contact with the enlarged end 212 of the shaft 200.

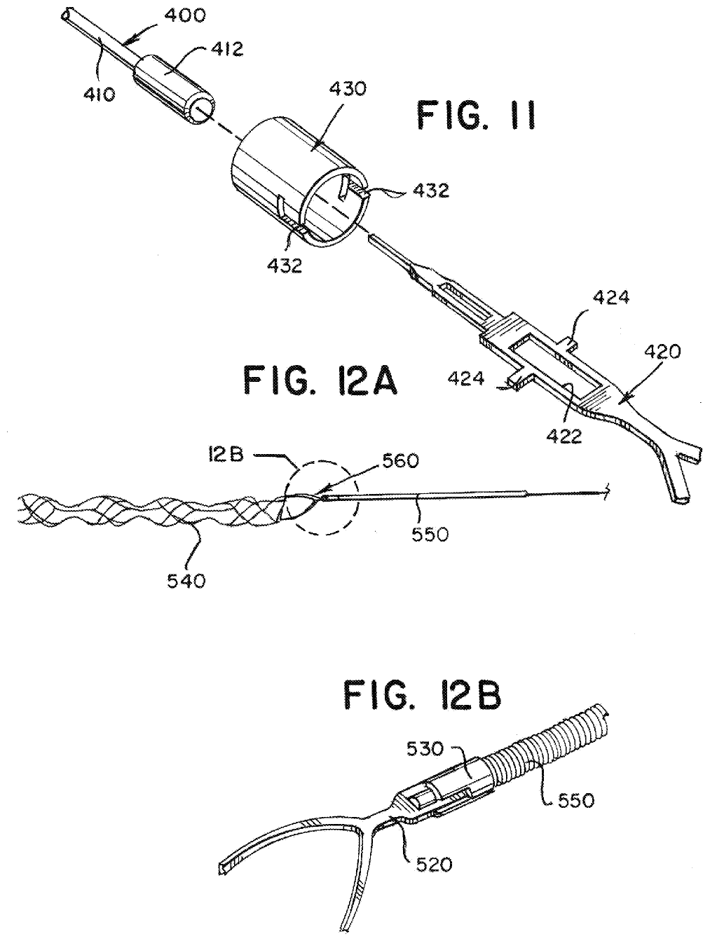

As shown in FIG. 10A, in some embodiments, an exemplary joint assembly can include a shaft 300, a proximal strut 320 including a proximal strut slot 322, and a locking collar 330. In some embodiments, the shaft 300 can include a main body 310 and a shaft hook 302. As shown in FIG. 10B, when assembled, shaft hook 302 of shaft 300 can be hooked into proximal strut slot 322. As shown in FIG. 10C, assembly can further include repositioning locking collar 330 such that it at least partially covers shaft hook 302 of shaft 300 and proximal strut slot 322 of proximal strut 320. This can prevent the shaft hook 302 from unhooking from the proximal strut slot 322. Shaft hook 300 can be formed by heat set or cut to shape. In some embodiments, collar 330 fully covers the shaft hook 302 ensuring the hook is atraumatic to the wall of vessel in which the joint assembly is inserted.

As shown in FIG. 11, in some embodiments, an exemplary joint assembly can include a shaft 400, a proximal strut 420, and a locking collar 430. Shaft 400 can include a main body 410 and an enlarged end 412. In some embodiments, the proximal strut 420 can further include a proximal strut slot 422 and proximal strut arms 424 protruding from proximal strut 420 on either side of proximal strut slot 422. Locking collar 430 can further include locking slits 432. In some embodiments, during assembly, the proximal strut 420 can engage locking collar 430 such that the proximal strut arms 424 are inserted into and rotated to lockingly engage locking collar slits 432. Examples can also include bayonet-type mounting.

FIGS. 12A and 12B show an exemplary joint assembly integrally joined to an endovascular device. In some embodiments, joint assembly 560 can be integrally joined to an endovascular device between a clot engaging portion 540 and an elongated shaft 550. Examples of endovascular devices can include a stentriever, thrombectomy device, coil retriever, equivalents thereof now known or later discovered, or combinations thereof. The joint assembly can include a proximal strut 520 and a locking collar 530.

The descriptions contained herein are examples of embodiments of the invention and are not intended in any way to limit the scope of the invention. As described herein, the invention contemplates many variations and modifications of the joint assemblies, including varied positioning of the shaft, proximal strut, and collar, utilizing any of numerous materials for each element or member, incorporation of additional elements or members, for example. These modifications would be apparent to those having ordinary skill in the art to which this invention relates and are intended to be within the scope of the claims which follow.

1. An endovascular device, comprising:
a shaft comprising a main body and an enlarged end;
a locking collar comprising a proximal face and a distal face, and at least partially covering the enlarged end of the shaft; and
a proximal strut comprising a cross-shaped member, wherein the cross-shaped member engages the proximal face of the locking collar.

2. The endovascular device of claim 1, wherein the locking collar comprises a first side and a second side, wherein a first slit is formed from the distal face of the locking collar into at least a portion of a first side of the locking collar and a second slit is formed from the distal face of the locking collar into at least a portion of a second side of the locking collar.

3. The endovascular device of claim 2, wherein the first slit and the second slit are tapered.

4. The endovascular device of claim 1, wherein a first slit is formed from the distal face of the locking collar into at least a portion of a first side of the locking collar and a polygonal aperture is formed in a second side of the locking collar.

5. The endovascular device of claim 1, wherein the cross-shaped member lockingly engages the proximal face of the locking collar.

6. The endovascular device of claim 1, wherein the locking collar has an ovoid shape and the cross-shaped member is passed through the locking collar from the distal face to the proximal face.

7. The endovascular device of claim 6, wherein the cross-shaped member is rotated 90° to engage slits on the proximal face.

8. The endovascular device of claim 1, further comprising an expandable cage, wherein the expandable cage is configured to engage a blood clot within vasculature.

9. A method of assembling an endovascular device comprising the steps of:
providing a shaft comprising:
a main body and an enlarged end;
a locking collar comprising a proximal face and a distal face and engaging the enlarged end of the shaft; and
a proximal strut comprising a cross-shaped member, wherein the cross-shaped member engages the proximal face of the locking collar;
sliding the locking collar onto the main body of the shaft;
positioning at least a portion of the proximal strut proximate the enlarged end of the shaft;
repositioning the collar such that it at least partially covers the enlarged end of the shaft and is adjacent the cross-shaped member; and
passing the cross-shaped member through the locking collar from the distal face to the proximal face.

10. The method of claim 9, wherein the cross-shaped member lockingly engages with the locking collar.

11. The method of claim 9, further comprising rotating the cross-shaped member 90° to engage with slits on the proximal face of the locking collar.

12. The method of claim 9, wherein the shaft, the proximal strut, the cross-shaped member, and the locking collar form a joint assembly.

13. The method of claim 9, further comprising integrally joining the joint assembly to an endovascular device between a clot engaging portion and an elongated shaft.

14. The method of claim 9, further comprising:
forming a first slit in a distal face of the locking collar into at least a portion of a first side of the locking collar; and
forming a second slit from the distal face of the locking collar into at least a portion of a second side of the locking collar.
